(19) United States
(12) Patent Application Publication
(10) Pub. No.: US 2009/0035789 A1
(43) Pub. Date: Feb. 5, 2009

(54) MONITORING ENZYME MIXTURES

(76) Inventors: Fred Rowell, Chester-Le-Street (GB); Latha Sundar, South Shields (GB)

Correspondence Address: QUARLES & BRADY LLP, 411 E. WISCONSIN AVENUE, SUITE 2040, MILWAUKEE, WI, 53202-4497, US

(21) Appl. No.: 12/278,702

(22) PCT Filed: Feb. 8, 2007
(86) PCT No.: PCT/GB2007/000430

§ 371 Date: Aug. 7, 2008

Related U.S. Application Data
(63) Continuation of application No. 11/349,261, filed on Feb. 8, 2006, now abandoned.

(30) Foreign Application Priority Data
Feb. 8, 2006 (GB) 0602518.3

(51) Int. Cl. G01N 33/573; C12Q 1/00; C12Q 1/37; C12Q 1/34; C12Q 1/40; C12M 1/00

(52) U.S. Cl. 435/7.4; 435/4; 435/23; 435/18; 435/22; 435/288.7

(57) ABSTRACT

This invention provides a method for simultaneously detecting the presence of at least two enzymes in a sample, said method comprising the steps of; i) providing a first substrate for a first enzyme, said first substrate being labeled with a first fluorophore, ii) providing a second substrate for a second enzyme, said second substrate being labeled with a second fluorophore, iii) exposing the labeled substrates to the sample to allow the first and second enzymes present in the sample to interact with respective first and second fluorophore-labeled substrates to form respective first and second fluorophore-labeled substrate fragments; and, detecting the presence of said fluorophore-labeled substrate fragments.

### THE FIELD OF THE INVENTION

The present invention relates to methods for monitoring for the presence of at least two enzymes in a sample. The enzymes can be considered mixtures of enzymes. In embodiments of the invention, the method comprises monitoring of enzyme mixtures on a continuous or near-continuous basis. The methods of the present invention can also be used to determine the concentration of an enzyme which is comprised within a mixture of enzymes. Also included in the present disclosure are products and apparatus for use in the method.

### BACKGROUND TO THE INVENTION

Enzymes of various types are widely used throughout a variety of industries such as the pharmaceutical industry, biotechnology industry, soap and detergent industry and the food industry. It has been observed that inhalation of enzymes released into the workplace atmosphere can cause deleterious health effects on exposed workers resulting from respiratory sensitisation. Statutory exposure limits have been identified such as for the proteolytic enzyme subtilisin and related enzymes, and it is a statutory requirement that the work place exposure limit (WEL) of 40 ng/m³ for this class of enzyme is enforced [1]. In order to conform to this requirement it is necessary to monitor for exposure at regular intervals or on a continuous basis for extended periods.

Systems for monitoring airborne enzymes currently consist of capturing the enzyme from the air, extracting the captured enzyme into solution and analysing the resulting solution for the constituent enzymes. The most common method for capture consists of passing air though a filter and analysis is largely based on spectrophotometric or spectrofluorescent methods in which the enzyme hydrolyses molecules of labelled substrate in solution [2], or in an immunoassay format such as ELISA [3]. The combination of capture onto filters followed by complex extraction and analysis cannot be developed to produce a sensitive, reagentless and continuous method for near real time monitoring of airborne enzymes and the results obtained represent time averaged values since intermittent release of enzymes cannot be monitored.

A method has been described that combines capture from air via impaction onto the surface of a cyclone and immediate analysis of the captured enzyme from the washed surface of the cyclone. The latter is achieved using fluorescent-labelled substrate specific for the enzyme in question that is immobilised onto a solid phase support contained within a fixed bed or bioreactor. Passage of the enzyme though the bioreactor results in partial digestion of the substrate and detection of the fluorescently-labelled fragments downstream [4]. This system allows continuous and near real time monitoring of a single enzyme which may be present in the atmosphere [5].

To date no system has been described that can simultaneously monitor mixtures which comprises at least two airborne enzymes on a continuous basis and in near real time. It is an aim of embodiments of the present invention to provide a method for monitoring enzyme mixtures for use on a continuous basis or for monitoring at frequent intervals.

### SUMMARY OF THE INVENTION

It is an aim of the present invention to provide an improved method of monitoring enzymes, which is suitable for use on a continuous basis or for monitoring at frequent short intervals. In particular, the methods and apparatus described herein can monitor the presence of a plurality of enzymes in a sample quickly and without the need for lengthy analysis steps. The methods and apparatus described have applications in various fields e.g. the monitoring of airborne enzymes in places in which the presence of such enzymes could have a deleterious effect on the health of personnel working in that area.

The method herein described enables the simultaneous qualitative and/or quantitative monitoring of a sample for the presence of at least two enzymes to be undertaken.

According to an aspect of the invention there is provided a method for the simultaneous detection of at least two enzymes in a sample, said method comprising the steps of;
(a) exposing (i) a first substrate for a first enzyme, the first substrate being labeled with a first fluorphore and (ii) a second substrate for a second enzyme, the second substrate being labeled with a second fluorphore to the sample to allow the first and second enzymes present in the sample, if present, to interact with respective first and second fluorophore-labeled substrates to form respective first and second fluorphore-labeled substrate fragments; and
(b) detecting the presence of the fluorphore-labeled substrate fragments.

In one embodiment, the method of the present invention is a method for detecting the presence and/or concentration levels of air-borne enzymes in an environment in which enzymes are produced and/or used, for example a laboratory or a factory floor.

In a further aspect of the present invention, there is provided a vessel for use in the simultaneous detection of the presence of at least two enzymes in a sample, the vessel comprising a first substrate for a first enzyme, the first substrate being labeled with a first fluorphore, and a second substrate for a second enzyme, the second substrate being labeled with a second fluorphore.

In a further aspect of the invention, there is provided an apparatus for use in the simultaneous detection of at least two enzymes in a sample, said apparatus comprising a vessel comprising a first substrate for a first enzyme, said substrate being labeled with a first fluorophore and a second substrate for a second enzyme, said second substrate being labeled with a second fluorophore, said apparatus further comprising detecting means for detecting fluorescent substrate fragments.

Also included in the present invention is the use of an apparatus described herein for the detection of at least two enzymes in sample. The use can be for the detection of the presence of air-borne enzymes. The use can also quantify the levels of at least two enzymes present within a sample of air.

### DETAILED DESCRIPTION OF THE INVENTION

In one aspect the present invention provides a method for the simultaneous detection of at least two enzymes in a sample, said method comprising the steps of;
(a) exposing (i) a first substrate for a first enzyme, the first substrate being labeled with a first fluorphore and (ii) a second substrate for a second enzyme, the second substrate being labeled with a second fluorphore to the sample to allow the first and second enzymes present in the sample, if present, to interact with respective first and second fluorophore-labeled substrates to form respective first and second fluorphore-labeled substrate fragments; and
(b) detecting the presence of the fluorphore-labeled substrate fragments.

The present method is for the simultaneous detection of the presence of a plurality of enzymes in a sample. As used herein, the term “simultaneous” refers to the detection of the presence of at least two enzymes at the same time or substantially at the same time, for example, the detection of the at least two enzymes takes place within a single operation of the method. The method can relate to the detection of a plurality of enzymes within the same sample, which does not require any additional input from a user between detection of the first enzyme and detection of the second enzyme.

It will be apparent that whilst the method is for the detection of at least two enzymes in a sample, in certain embodiments, on certain occasions the sample may contain only one or no enzymes. However, the method will be suitable for detecting at least two enzymes if they are present in the sample.

The method described herein refers in the main part to the monitoring of two enzymes. It will be apparent to the skilled person that the method of the present invention can also be used to detect the presence and/or quantify the levels of more than two enzymes e.g. 3, 4, 5, 6 or more. If the method is used to detect more than two enzymes in a sample, a corresponding number of labeled substrates are provided. For example, if the method is used to detect three enzyme types in a sample, three labeled substrates are provided, each substrate corresponding to an enzyme i.e. for each enzyme to be detected, there is provided a labeled substrate which interacts with it detectably.

In one embodiment, the method is for detecting the presence of at least two air-borne enzymes.

In this embodiment, the method may comprise first mixing a first sample of air with a liquid to form a solution which forms the sample which contacts the substrates. In this embodiment, the method may comprise as sucking the first sample of air into an apparatus which collects particles e.g. enzyme particles, which are carried in the air. The method may further comprise supplying the apparatus with a liquid which mixes with the collected particles to produce the solution. In one embodiment, the apparatus is a tapered cone. In one embodiment, the method can comprise monitoring the rate of air flow into the apparatus and, if required, adjusting the flow of air to ensure that it generally mimics the intake rate of air when a person inhales. This enables a comparison to be made between the data obtained by covering out the method, and the likely amount of air-borne enzymes taken in by an individual during normal breathing.

In one embodiment, the liquid is added from a reservoir to the apparatus on a continuous basis. In this embodiment, the level of liquid in the reservoir is kept constant by continuous topping up. In an alternative embodiment, the level of the liquid in the reservoir is not maintained constantly. Instead, the method comprises emptying the reservoir and re-filling it on a regular basis e.g. after each time a first sample of air is taken or for example after a certain number of air samples have been taken e.g. two, three, four or five or more air samples. The reservoir may be emptied and refilled after a cycle of the method has taken place. In one embodiment, when being used, the reservoir can be emptied and re-filled about every 10 to 20 minutes e.g. about every 10, 11, 12, 13, 14, 15, 16, 17, 18, 19, 20 or more minutes.

In one embodiment, the method can comprise conveying the sample from the apparatus described above to a reaction area in which the sample is brought into contact with the fluorophore labeled substrates. The reaction area may comprise a reaction vessel or a plurality of vessels. The method comprises bringing the sample into contact with the substrates for a time sufficient to permit a reaction between the enzymes and their corresponding substrates to form a reaction product e.g. a labeled substrate fragment. The reaction product can then detected. In one embodiment, the step of detecting comprises use of a detecting means which detects the level of signal emitted by the fluorophore attached to the substrate fragment. In one embodiment, the detecting means is a spectrophotometer which can then be used to determine the presence and/or quantities of enzyme(s) present in the sample. In one embodiment, the detecting means includes optical fibres attached to a flow cell and which are linked to pre-tuned light sources (excitation) and multiple photon multiplier tubes (pmt-detection systems).

In some embodiments, the sample is contacted with the first and second substrates for a reaction time of at least about 1 sec, 5 sec, 10 sec, 15 sec, 20 sec, 25 sec, 30 sec, 35 sec, 40 sec, 45 sec, 50 sec, 55 sec, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, or 60 min. Preferably, the sample is contacted with the labeled substrates for a reaction time at least about 1 minute, at least about 5 minutes, at least about 10 minutes, or at least about 15 minutes.

Typically, the substrates used in the present method are selected from among those with which the enzyme to be monitored reacts/digests. As used herein, the term “substrate” refers to a substance which interacts with an enzyme in such a way as to cause a change in the substance. For example, the enzyme can react with the substrate and/or can digest the substrate into fragments. The substrates used in the present invention are labeled in such a way so as to enable detection of the interaction between the enzyme and the substrate. In particular, the label is a fluorophore.

In a preferred embodiment of the invention the substrate is a protein or polypeptide. The terms “polypeptide”, “peptide” and “protein” are used interchangeably herein to refer to polymers of amino acids of any length. The polymer may be linear or branched, it may comprise modified amino acids or amino acid analogs, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labelling component.

Amino acid substitutions can range from changing or modifying one or more amino acids to complete redesign of a region, such as the variable region. Amino acid substitutions are preferably conservative substitutions that do not deleteriously affect folding or functional properties of the peptide. Groups of functionally related amino acids within which conservative substitutions may be made are glycine/alanine; valine/isoleucine/leucine; asparagine/glutamine; aspartic acid/glutamic acid; serine/threonine/methionine; lysine/arginine; and phenylalanine/tryosine/tryptophan. Polypeptides used in this invention may be in glycosylated or unglycosylated form, may be modified post-translationally (e.g., acetylation, and phosphorylation) or may be modified synthetically (e.g., the attachment of a labeling group).

In one embodiment, the first substrate is selected from the group consisting of gelatin, porcine thyroglobulin, collagen, an immunoglobulin and bovine serum albumin. In one embodiment, the second substrate is selected from the group consisting of gelatin, porcine thyroglobulin, collagen, an immunoglobulin a bovine serum albumin. In an embodiment, the first substrate is gelatin. The first and second substrates may be the same or may differ from each other.

The fluorophores used in the methods of the invention to label the substrates are selected to be detectable by normal fluorometric methods. Furthermore, the fluorophores are particularly chosen so that each fluorophores' spectral properties do not interfere with the fluorescent signals of other fluorophores co-released via the reaction of other enzymes present in the mixture with their specific substrates. Preferably the selected fluorophores are not influenced by variations in ambient conditions such as pH level. In one embodiment, the first and second fluorophore are independently selected from the group consisting of fluorescein and its derivatives, rhodamine, Texas Red®, cresol violet and lucifer yellow. Preferably, the fluorophores used to label the first substrate is different to the fluorophore used to label the second substrate.

The labelled substrates are in many cases advantageously carried upon a suitable support, for example, although not limited to, glass beads, fibrous cellulose or sol gel particles. Supports, for example, solid phase supports, are well known in the art and can be biological, non-biological, organic, inorganic, or a combination of any of these, provided that they do not interfere with the reaction between the labeled substrates and any enzymes present in the sample. Examples of solid support which can be used in the present invention include, although are not limited to, particles, beads, strands, precipitates, gels, sheets, tubing, spheres, containers, capillaries, pads, slices, films, plates, slides. Particle size may range from 100 nm to 100 μm in diameter.

The labeled substrates can be attached to the support by various means known in the art. For example, the substrate can be covalently attached to the support surface. In one embodiment, the substrate can be linked via direct absorption to the support surface.

In a further preferred embodiment of the invention, the support comprises beads that are magnetisable. In one embodiment, the support comprises a plurality of silica particles on to which the substrate(s) are attached. Other types of solid support are known in the art and encompassed by the present invention.

In some embodiments, the substrate may not be on a support. For example, in the case of cellulose, in view of its insolubility in water, the substrate may be used as solid particles without such support.

The method is used to monitor for the presence of at least two enzymes in a sample. Examples of enzyme types which may be detected by application of the methods of the present disclosure, include, but are not restricted to, members of the protease, cellulase, lipase, amylase or collagenase families. In one embodiment, cellulases can be monitored using labelled cellulose as one of the substrates and/or collagenases can be monitored using labelled collagen as one of the substrate.

In an embodiment, one of the at least two enzymes is a protease. In an embodiment, the protease can be selected from the group consisting of a subtilisin-type enzyme, trypsin, papain, esperase and alcalase.

In one embodiment of the invention, the first substrate is gelatin. In this embodiment, the first enzyme which is to be detected and/or quantified is a subtilisin-type enzyme. In one embodiment, the second substrate is selected from starch, amylose and amylopectin. In this embodiment, the second enzyme is α-amylase. Various combinations of enzymes may be detected by the methods of the present disclosure.

If the sample contains enzyme(s) which interact with at least one of the substrates, once the sample is exposed to the substrates, a reaction between the enzyme(s) present and the corresponding substrate takes place. In one embodiment, reaction between the enzyme and the substrate results in formation of fragments of the substrate. The labelled substrate fragments released from each vessel following exposure to the sample containing the enzymes are monitored downstream using spectrofluorimeter(s) tuned to a particular fluorophore or plurality of fluorophore. The method may further comprise the step of tuning the spectrofluorometer to detect the first and second fluorophores.

As described above, in one application of this method, the sample is derived from a first sample of air. One application of the present method is in the work place, where there are statutory limits of employees' exposure to air-borne enzymes. In particular, the method may be carried out in areas in which enzymes are being used to produce for example washing powders and food stuffs.

The method can be put into practice in various ways. In one embodiment, the method is performed on continuous basis by conveying the sample continuously into a collector containing a solution. The sample then contacts the labeled substrates in a reaction area which contains, for example, a vessel or plurality of vessels.

As described above, in an embodiment in which the sample is derived from a first sample of air, the air is placed in solution. In one embodiment, the solution is a buffer which is chosen on account of it having a certain pH for passing through the system. Thus, in one embodiment, the solution is an aqueous buffer. In one embodiment, the aqueous buffer is, for example, phosphate buffered saline.

In one embodiment, the reproducibility of the fluorescence signals is further enhanced by the inclusion of a small quantity of a detergent in the solution. In an embodiment, the method comprises providing a detergent to the solution. The detergent can be, for example, Tween 20. The detergent may be utilised to reduce non-specific binding between released fragments and surfaces within the flow injection analysis system.

In an embodiment of the method of invention, the substrates are provided within a reaction vessel such that enzyme(s) contained within the sample simultaneously contact the first substrate and the second substrate. For example, in an embodiment in which the substrates are supported on beads, the reaction vessel contains a heterogenous mixture of beads, some of which support a labeled first substrate and some of which support a labeled second substrate. In an embodiment, the reaction vessel(s) is a column. In one embodiment, the reaction vessel(s) can be small i.e. sized to be easily carried to, or installed at the site to be monitored.

In an alternative embodiment of method of the invention the enzymes derived from the sample sequentially contact the first substrate and the second substrate. For example, the reaction vessel contains stratified homogeneous layers of the different substrates. Thus, the reaction vessel can comprises layers of support, each layer containing essentially one type of substrate. As a result, the sample containing the mixture of enzymes comes into contact with a layer of e.g. supported first substrate followed by a layer of supported second substrate.

Alternatively, the method comprises providing a plurality of reaction vessels in series, each reaction vessel containing a single type of labeled substrate. As a result, the sample contacts a first substrate and then contacts a second substrate.

Thus, in one embodiment the invention utilizes at least two reaction vessels which are positioned in series, each containing different substrates. The vessels can be linked with a conduit which permits the sample to be conveyed between the vessels. In this embodiment of the invention, the first vessel can contain the first substrate and the second vessel can contain the second substrate. Further reaction vessels containing further substrates for other enzymes may be provided.

In one embodiment, the invention can comprise providing a mixture of different vessel types contacting the sample sequentially with the first substrate and second substrate via a plurality of reaction vessels.

In embodiments in which it is expected that the sample being analysed contains a protease, one of the first or second substrates is a substrate for the protease. In this embodiment, it is preferable to ensure that the sample contacts the substrates sequentially. Even more preferably, the substrate for the protease is positioned adjacent to the detecting means. It has been found that fragments of the digested protease substrate can stick to the support e.g. the solid phase support, which bears the other substrate(s). Thus, for example, when the method is for detecting a mixture of enzymes including α-amylase and a subtilisin-type enzyme, the substrate for the subtilisin-type enzyme is preferably positioned adjacent to the detecting means i.e. downstream in relation to the other substrate(s). Thus, the reaction vessel containing a substrate for a protease enzyme is positioned downstream from the reaction vessel(s) containing substrates for other types of enzymes.

The detection of the fluorescently-labelled substrate fragment can be undertaken on site (e.g at a factory), if the appropriate equipment, for example a spectrophotometer is available. Alternatively the reaction vessel can be taken to a laboratory where the analysis is undertaken.

The method may further comprise calibrating the results by comparing the quantity of the at least two enzymes present in the sample with a standard.

According to a further aspect of the invention there is provided a vessel for use in the simultaneous detection of at least two enzymes in a sample, said vessel comprising a first substrate for a first enzyme, said first substrate being labeled with a first fluorophore, and a second substrate for a second enzyme, said second substrate being labeled with a second fluorphore.

In an embodiment of the invention, the labeled substrates are carried upon a suitable support, as described above. In one embodiment, the support is or comprised from, for example glass beads, cellulose e.g. fibrous cellulose, silica e.g. silica particles or sol gel particles. If the support comprises particulate matter, the particle size may range from about 100 nm to about 100 μm in diameter.

In an embodiment of the invention, when the support is for example beads or silica particles, the reaction vessel contains a heterogeneous mixture of beads or particles. That is to say, a portion of the beads or particles support the first substrate, whilst a portion of the beads or particles support the second substrate and there is no discernable order to the arrangement of the beads. In an alternative embodiment of the invention the reaction vessel contains stratified homogenous layers of the substrates.

According to a further aspect of the invention there is provided apparatus for use in the simultaneous detection of at least two enzymes in a sample, said apparatus comprising a vessel comprising a first substrate for a first enzyme, said substrate being labeled with a first fluorophore and a second substrate for a second enzyme, said substrate being labeled with a second fluorophore, said apparatus further comprising detecting means for detecting fluorescent reaction products. In one embodiment, the first and second fluorophore are different fluorophores. The fluorophores may be as described herein.

In an embodiment, the first and second substrates can form stratified layers within the vessel. In one embodiment, one of the first or second substrates is a substrate for a protease. In this embodiment, the vessel comprises a layer of protease substrate at a position which is closer to a detecting means than the other substrates. Thus, in one embodiment, the substrate for a protease enzyme is located adjacent to said detecting means i.e. downstream from the other substrates.

Preferably the detecting means is a spectrophotometer.

According to a still further aspect of the invention there is provided apparatus for the simultaneous detection of at least two enzymes in a sample, said apparatus comprising, in use, a first vessel comprising a first substrate for a first enzyme, said first substrate being labeled with a first fluorophore, said first apparatus being connectable to a second vessel, wherein the second vessel comprises a second substrate for a second enzyme, said second substrate being labeled with a second fluorophore, said apparatus further comprising detecting means for detecting fluorescent reaction products. The reaction products are produced from interaction between an enzymes and its fluorophore labeled substrate.

In a preferred embodiment of the apparatus, the substrate in the second vessel is a protease, and the second vessel is located adjacent to the detecting means.

In one embodiment, the apparatus comprises more than two reaction vessels e.g. three, four, five or more reaction vessels. Each reaction vessel may comprise a fluorophore labeled substrate for an enzyme.

Preferably the detecting means is a spectrophotometer.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

The invention will now be described by way of example only and with reference to the accompanying drawings in which;

FIG. 1a; calibration plot for subtilisin-type protease (Savinase™) enzyme alone.

FIG. 1b; calibration plot for an α-amylase enzyme (Termamyl™) alone.

FIG. 1c; calibration plot for α-amylase in an equi-concentration mixture of the subtilisin-type protease and α-amylase.

FIG. 2: Schematic Representation of a monitoring system of the embodiments of the present invention

### EXAMPLES

### Example 1

A system is described which is able to monitor airborne concentrations of a variety of air-borne enzymes including for example subtilisin-type enzymes in the workplace atmosphere on a continuous basis. Sampling comprises two stages: using a sampling head that is designed to mimic human respiration at approx. 1 m s⁻¹ at a sampling rate of 600 l min⁻¹. In the second stage, the captured particles are deposited by impaction from the air stream onto the inner surface of a cyclone that is continuously washed with a jet of buffer solution. Deposited particles are then washed into a reservoir from which samples are taken every 5-6 min and injected automatically into a continuous flow injection analysis system. Proteolytic enzyme in the sample passes through a bioreactor maintained at about 40° C. In one embodiment, this contains a cellulose solid phase matrix on which is covalently immobilised Texas Red-labelled gelatin as substrate. The bioreactor can also contain a second substrate for an additional enzyme. Alternatively, a separate bioreactor containing a second substrate is connected to the first bioreactor (not shown).

In other embodiments of the invention, the substrate can be immobilized on silica particles. The passing enzyme(s) partially digests the substrate(s) releasing fluorophore(s) that are detected down stream in a flow cell coupled to a fluorimeter. The system is calibrated using enzyme standards and the intensity of the resulting peaks from the ex-air samples is converted to airborne concentrations using a mathematical model programmed into a PC. The system has a limit of detection of 4.8 ng m⁻³ and a dynamic range of 5-60 ng m⁻³. The within assay precision (RSD) is 6.3-9.6% over this range. The within batch precision is 20.3% at 20 ng m⁻³ and the corresponding between batch value is 19.5%. The system has been run for periods up to 8 h and for up to 4 h and the values obtained compared with time-averaged values obtained from a conventional Galley sampler and in-house analysis when reasonable agreement of the results was observed. The stability of the system over 21 days of continuous use with standards injected periodically was studied. Linearity was observed for all the standard plots throughout. At the end of 21 days, after a total exposure equivalent to 2395 ng ml⁻¹ of Savinase, the signal due to the 5.0 ng ml⁻¹ standard was still easily detectable.

### Experimental Apparatus

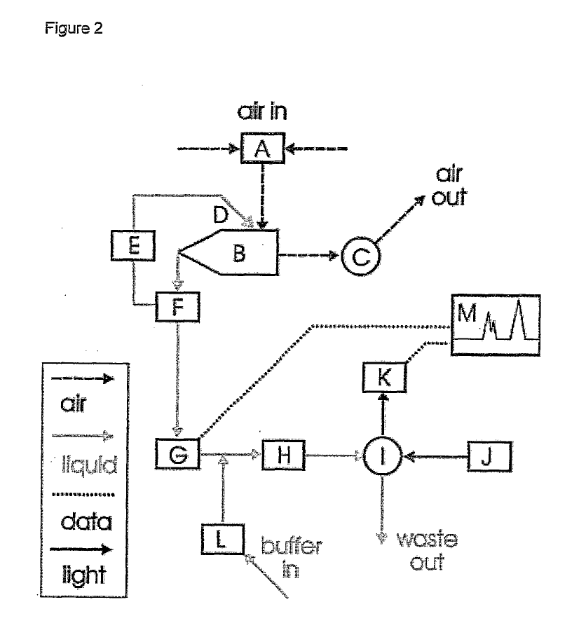

The system is shown in FIG. 2. It includes a sampler head comprising a pair of circular metal plates (A) attached via plastic tubing to a glass cyclone (B). Air is sucked through A and B using a commercial vacuum cleaner (C). The rate of airflow was monitored using an orifice plate and adjusted as needed. A needle (D) was attached to B and buffer solution was pumped through its orifice using an adjustable peristaltic pump (E). As air flowed through the cyclone this jet of water covered the inner surface of the cyclone and flowed into a reservoir (F) from which it was recycled via E back into the cyclone. The cyclone is described in more detail below.

Following air sampling, known volumes of buffer are taken periodically from F into an automated flow injection system (G) attached to a bioreactor or plurality of bioreactors within a thermostatically controlled column heater (H). Downstream is located an optical flow cell (I) equipped with two optical fibres attached to a light source (J) and a spectrophotometer (K). Phosphate buffered saline is passed through the FIA system using a peristaltic pump (L) and the signal from K recorded using a lap top PC (M). This PC also controlled the automated flow injection system. In both cases the programmes used were those supplied by the manufacturer of these two systems (FIAlab, Medina, Wash., USA).

In an alternative method, the reservoir is not topped up continuously to keep the reservoir levels constant. Instead, samples are taken for about 12 minutes as described above. The sample is then analysed and the reservoir is flushed out completely and re-filled. A new cycle of obtaining samples and analysis is then begun. This method has the advantage of not requiring a complex system of calculating the effect re-filling the reservoir has on enzyme concentration. As a result, this method can be simpler to carry out.

The automated flow injection system (G) was model 3500 from FIAlab Instruments. The column heater (H) was an Eppendorf CH500 heater (Phenomenex, Macclesfield, Cheshire, UK), which was set to operate at an exit buffer temperature of 40° C. at a flow rate of 0.7 ml min⁻¹. The flow cell (I) was model X from FIAlab who also supplied the two stainless steel-coated optical fibres (P400-2), the LS-1 tungsten halogen light source and the S2000 spectrometer (J). The peristaltic pump (K) was model P-1 from Pharmacia (Milton Keynes, Buckinghamshire, UK).

### Design of the Cyclone

A glass cyclone of dimensions (40 mm diameter, 20 mm outlet, 30 10 mm rectangular inlet, and a main body of 80 mm length and cone of 80 mm length) was constructed which had three pre-drilled holes in the air inlet tube to allow up to three positions of the needle for the introduction of the recycling fluid. These holes were positioned at the centre of the inlet tube and 6 mm either side. A stainless steel hypodermic needle was bent at a right angle (10 mm from tip) to allow the recycling fluid to be pumped into the cyclone.

The cyclone was tested to measure its grade efficiency by loading fine dust through the cyclone inlet and measuring the difference in particle size and concentration of the dust inputted into the cyclone and that of the dust exiting the cyclone using an aerodynamic particle sizer (APS).

### Reagents and Materials Used in the Flow Injection Analysis System

The composition of buffers, sources of enzymes, and chemicals and other materials used in the flow injection system and in bioreactor production are those described previously⁵⁻⁸. Gelatin type B (about 75 bloom) was from Sigma (Poole, Dorset, UK). Texas Red® succinimidyl ester was from Molecular Probes (Eugene, Oreg., USA). The serine-type proteolytic enzymes studied were Purafect (Genencor International Inc., Rochester, USA) and Savinase from Novozymes, Bagsvaerd, Denmark, who also supplied the lipase and cellulase enzymes. Papain was from Papayer Latex and supplied by Sigma.

### Synthesis of Gelatin-Texas Red Conjugate.

One of the substrates is a gelatin-Texas Red conjugate. Gelatin (0.105 g) was dissolved in sodium bicarbonate buffer (10 ml of 0.1 M at pH 8.3) with gentle heating (40 1C). Texas Red® succinimidyl ester (5 mg) was dissolved in 500 ml of dimethyl sulfoxide. This mixture was then was then added to the dissolved gelatin solution. The container was covered with tin foil and mixed on a rotator mixer for 1 h at room temperature. When stored at 4° C., this solution of labelled gelatin is stable for approximately 3-4 weeks.

### Synthesis of Cellulose-Gelatin-Texas Red Solid Phase.

Medium cellulose fibre (5 g) was rinsed in deionised water and centrifuged (2000 rpm for 6 min) prior to removing the supernatant solution. This was repeated three times after which the cellulose was filtered under vacuum using a size 2 sintered glass funnel (BDH Poole, Dorset). Sodium m-periodate solution (80 ml of 0.5 M) was added to the damp cellulose and the mixture rotated for 210 min at room temperature. The activated cellulose was then filtered and washed with 2 l deionised water as above. The activated cellulose was stored at 4° C. in deionised water containing 0.02% sodium azide, and was stable for 3-4 weeks.

Activated cellulose (2.5 g after filtration) was suspended in 0.1 M anhydrous dibasic sodium phosphate buffer (65 ml). To this was added the gelatin-Texas Red conjugate (10 ml). The container was coated in aluminium foil and rotated for 45 min at room temperature prior to the addition of sodium cyano-borohydride (250 mg). The tube was replaced on the rotator and left to mix overnight at room temperature. After coupling, the cellulose-gelatin-Texas Red solid phase was filtered under vacuum as above and washed with 2 l of deionised water. The resulting cellulose-gelatin-Texas Red solid phase was also stable for 3-4 weeks when stored under refrigerated in deionised water containing 0.02% sodium azide.

### Development of the Flow Injection Analysis System

FIA conditions. The FIA buffer and that for the enzyme standards were phosphate buffered saline solution (PBST). This contained sodium chloride (7.2 g), anhydrous dibasic sodium phosphate (1.48 g), sodium dihydrogen orthophosphate monohydrate (0.5 g), sodium azide (0.5 g) and polyoxyethylene-sorbitan monolaurate (1.0 ml) (Tween 20^{s} ICI Americas, Inc.) per litre. The FIA flow rate was maintained at 0.7 ml min⁻¹ using small adjustments of the peristaltic pump. The sampling program was compiled using the FIALab for Windows version 5. The sampling volume used was 100 ml, total injection volume 180 ml at a rate of 12 mls⁻¹. A 590 nm filter was inserted into the path of the light source (J) and the spectrophotometer (K) was set at 612 nm.

### Results

### Design of the Air Sampling System

Testing of the cyclone design and its performance. The cyclone was tested across a range of airflows (nine different airflow from 176 to 732 l min⁻¹), and at 608 l min⁻¹ was shown to have a d50 of approximately 0.77 mm with an SD of 1.1%. The cyclone can be seen to efficiently capture at least 90% of the particles of 1.5 mm and above an air flow of B600 l min⁻¹. This establishes that the cyclone is capable of capturing the particle sizes as required by the project and of a similar size to the filters used in Galley samplers which have a 1.2 mm pore size.

The cyclone design was connected to an air sampling inlet system. This is designed to mimic human respiration when operating at an sampling rate of about 600 l min⁻¹ and at an inlet flow of about 1 m s⁻¹.

Within the analytical system, a new substrate was used to produce the required signals for one of the enzymes. Gelatin was conjugated with Texas Red® to produce a fluorescent label that was then covalently immobilised onto a cellulose support matrix. Gelatin was chosen as one of the substrates of the invention because it provides many sites for fluorescent conjugation and potential enzyme hydrolysis. Upon exposure to subtilisin-like protease enzymes, proteolytic cleavage of the immobilised gelatin occurred, resulting in fluorescent signals that were recorded by the spectrophotometer. Other substrates are also utilized in the present method.

The apparatus described above constitutes a near real-time system for determining the airborne concentration of for example enzymes in industry. The buffer chosen is suitable for maintaining the protease activity of the enzyme and the fluorophore signal. Its use also minimises the leaching of fluorophores, thus extending the lifetime of the bioreactor (data not shown).

The system has good within-assay precision and reasonable within and between batch variability. The variation stems mainly from inconsistent packing of the columns during manufacture of the bioreactors. This is currently performed manually but an automated system would probably lead to much improved batch consistency.

The system is relatively portable and has successfully been demonstrated over extended periods. The system also provides a near-continuous record of airborne concentrations of enzymes throughout the sampling period. The system is a near time system, with responses of 5 min and a capacity for continuous monitoring over 8 h periods under industrial conditions.

### Example 2

In an alternative embodiment, the method utilizes a different support for the substrates. Sol gel particles activated with glycidoxypropyltrimethoxysilane were treated with gelatin pre-labelled with Texas Red® to produce a solid phase substrate-coated solid phase support for subtilisin-type proteases (e.g Savinase™).

Other sol gel particles activated with aminopropyltrimethoxysilane were treated with starch labelled with fluorescein ethylene diamine to produce a solid phase substrate for α-amylase (e.g Termamyl™).

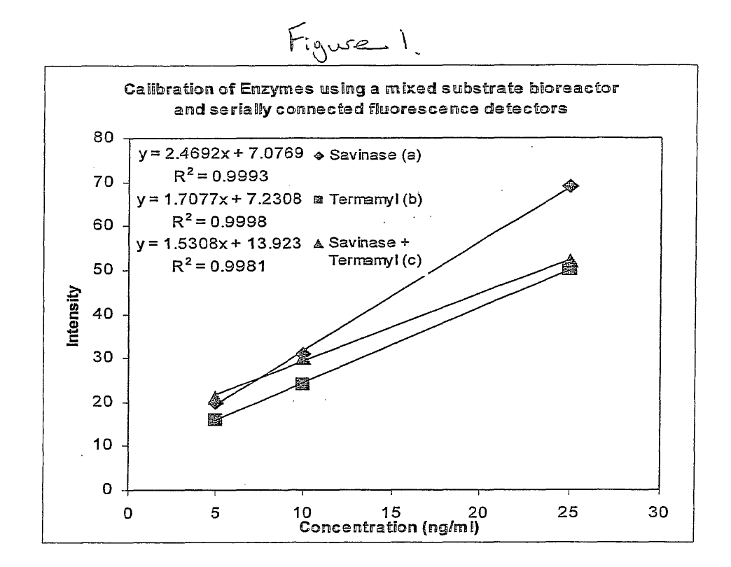

Equal masses of the two solid phase reagents were layered and then packed into a mini-column such that the bottom layer consisted of the particles coated with the substrate for the subtilisin-type enzyme and the upper layer consisted of the particles coated with the substrate for α-amylase. The mini-column was fed, at a rate of flow of 0.7 ml/min, with samples containing 5 to 25 ng/ml of mixtures of savinase and amylase or the individual enzyme component, in phosphate buffered saline (pH 7.4) with Tween 20 (0.1%). The samples were obtained using the cyclone described above. The mini-column was linked downstream to two spectrofluorimeters connected in series, the first set at fluorescein fluorescence (490 nm excitation, 535 nm emission) and the second at Texas Red® fluorescence (590 nm excitation, 620 nm emission), and the fluorescence intensities of the two fluorophores simultaneously and continuously monitored. A response time of 2 minutes was observed for either enzyme with sample repeats possible every 5 minutes. Linear signal/concentration standard curves were obtained for the single enzymes (FIG. 1a, protease only; FIG. 1b, amylase only) and for the mixture of the two enzymes over this concentration range (FIG. 1c, response for amylase shown) when injected into the column.

### Example 3

### Preparation of Fluorescent Substrates for Use in Packing Bioreactors for Alpha-Amylase

### Preparation of Fluorescein-Labelled Ethylene Diamine (EDA-FITC)

The material used were Fluorescein Isothiocyanate Isomer I (FITC), Sigma (F-7250), N,N-Dimethylformamide (DMF), Aldrich (31,993-7), 1, 4 Dioxane, Aldrich (27,053-9) and Ethylene diamine, Aldrich (E 2,626-6).

Take a clean dry 50 ml glass bottle and weigh accurately 39 mg of FITC. Dissolve FITC by adding 200 μl of DM. Add 4.8 ml of 1, 4 Dioxane to the FITC solution. The add 6.68 μl of Ethylene diamine to the FITC. Stir the EDA-FITC solution for 1 hr on a magnetic stirrer at room temperature by keeping it covered with aluminium foil. A dark orange colour precipitate should be formed.

After 1 hr stirring, remove the bottle and centrifuge at 3000 rpm for 3 minutes. Discard the supernatant in solvent waste and add 5 ml of 1, 4 dioxane, vortex and centrifuge at 3000 rpm for 3 minutes. Discard the supernatant in solvent waste and transfer the precipitate to a round bottom flask using minimal amount of 1, 4 dioxane. Evaporate the solvent by Rotary evaporating at 50° C. for approximately 3 hrs. Once the precipitate is dry, weigh it and transfer to a clean dry glass vial and store in a desiccator at 4° C. ensuring it is covered in foil.

### Activation of Labeled Ethylene Diamine (EDA-FITC)

The materials required are cyanuric chloride, Aldrich (C 9,550-1), labelled Ethylene diamine (EDA-FITC) (see ref 1 above), N,N-Dimethylformamide (DMF), Aldrich (31,993-7), Triethylamine, Fluka (90340) and phosphate buffered saline with azide, pH 7.4 (PBS with azide), (see SOP-Buffers 003)

Prepare 4.3 ml of 10 mg/ml of EDA-FITC solution by weighing 43 mg of EDA-FITC as prepared in 1 in a clean dry glass bottle and dissolving it in 4.3 ml of DMF. Weigh 21.5 mg of cyanuric chloride and add to the EDA-FITC solution. Add 20 ml of 1 mg/ml of Triethylamine solution prepared by adding 27.5 μl of Triethylamine in 20 ml of PBS with azide to the activated EDA-FITC solution. Vortex the mixture to ensure thorough mixing. A precipitate should form. Store the precipitate in dark at 4° C.

Coupling of Activated Labeled Ethylene Diamine (ACT-EDA-FIFC) to Starch Substrate (Amylopectin and Amylose can Also be Used)

The materials required are: starch soluble, Sigma (S-9765), phosphate buffered saline with azide, pH 7.4 (PBS with azide), activated EDA-FITC (see above) and sodium carbonate, Sigma (S-7795)

### Preparation of 0.5M Sodium Carbonate Solution:

Weigh 5.495 g of sodium carbonate in a 100 ml volumetric flask and dissolve using minimal amount of deionised water. Once dissolved, make up to the mark

### Coupling of Act-EDA-FITC to Starch Substrate

Weigh 0.5 g of soluble starch in a clean dry 25 ml glass bottle. Add 15 ml of PBS to the starch and heat it in a water bath approximately maintaining 60-70° C. with continuous stirring until a transparent suspension is formed. A thick jelly transparent suspension should be formed.

To the starch suspension add 2 ml of activated EDA-FITC (Ref 2 above) and vortex it to give a uniform suspension. Place it back in the water bath and continue heating (˜60-70° C.) by stirring continuously in dark. After 10 minutes add 200 μl of 0.5M Sodium carbonate solution and continue stirring at 70° C. for 1 hr in dark. Remove the suspension from the hot water bath after an hour and cool it to room temperature by placing it in a fume cupboard.

Once cooled, transfer the suspension to a dialysing tube and dialyse against deionised water maintained at 37° C. Continue dialysis until no fluorescence is observed in the deionised water. This should take approximately three to four days. Once dialysed, transfer the suspension to a 30 ml sterilin tube and store it in the fridge by wrapping it in a foil.

### Preparation of Aminopropyl Triethoxysilane (APTES) Coated Sol-Gel, Silica or Silica Gel Particles

The materials required are sol-gel particles, aminopropyl triethoxysilane (APTES), Sigma (A3648) and phosphate buffered sailine, pH 7.2 (PBS with azide)

Weigh approximately 10 g of dry sol-gel particles (125-180 um) in a 50 ml sterilin tube. Add 20 ml of PBS and vortex the particles. Centrifuge at 3000 rpm for 3 minutes. Discard the supernatant and add 2 ml of APTES and rotate for 2 hrs in a rotator. Remove it from the rotator and centrifuge at 3000 rpm for 3 minutes. Discard the supernatant in solvent waste and add 20 ml of PBS and vortex for 30 secs and rotate for 5 minutes. Repeat the washings 3 more times by following steps 5 and 6 i.e. remove it from the rotator and centrifuge at 3000 rpm for 3 minutes. Discard the supernatant in solvent waste and add 20 ml of PBS and vortex for 30 secs and rotate for 5 minutes. The APTES coated particles are ready for use. For storage, suspend the particles in minimal amount of PBS and store at 4° C.

### Immobilisation of Starch-EDA-FITC to APTES Coated Sol Gel and Similar Silica Particles

The materials required are APTES coated Sol-gel particles (see above), phosphate buffered sailine, pH 7.2 (PBS with azide) and starch-EDA-FITC (see above)

Weigh about 5 g of moist weight of APTES coated sol-gel particles (125-180 μm) in a 30 ml Sterilin plastic tube. Add 20 ml of PBS azide and vortex the particles. Add 5 ml of STARCH-EDA-FITC substrate and rotate overnight. Remove it from the rotator and centrifuge at 3000 rpm for 3 minutes. Discard the supernatant in solvent waste and add 20 ml of PBS and vortex for 30 secs and rotate for 5 minutes. Repeat the washings 3 more times by following steps of removing it from the rotator and centrifuging at 3000 rpm for 3 minutes and then discarding the supernatant in solvent waste and add 20 ml of PBS and vortex for 30 secs and rotate for 5 minutes. The starch substrate immobilised on sol-gel particles are ready for packing.

Store the immobilised starch particles in minimal amount of PBS azide at 4° C. and use it within 3 days of preparation. Pack about 0.8 g of wet derivatised particles into columns as described for gelatin-cellulose above and condition as for protease bioreactor columns.

### REFERENCES

1. EH40/2005 Workplace exposure limits, Environmental Hygiene Guidance Note EH40, HSE Books, ISBN 07176 29775
2. Rothgeb, T. M., Goodlander, B. D., Garrison, P. H. & Smith, L. A., J. Am. Oil Chemists Soc., 65, 806 (1988)
3. Miao, Z-H, Rowell, F. J., Reeve, R. N., Cumming, R. H., Journal of Environmental Monitoring, 2, 451-454 (2000)
4. Monitoring of Enzymes, PCT/GB96/03052
5. Tang, L. X., Rowell, F. J., Cumming, R. H., Annals Occupational Hygiene, 40, 381-389 (1996)
6. D. Sykes et al J. Aerosol Sci., 2000, 31, S90-91
7. L. X. Tang et al Analyst, 1995, 120, 1949
8. Tang, L. X., Rowell, F. J., Cumming, R. H., Anal. Proc. Incl. Anal. Comm. 1995, 32 519

Also included in the present disclosure is the subject matter of the following paragraphs:

1. A method for simultaneously detecting the presence of at least two enzymes in a sample, said method comprising the steps of;
providing a first substrate for a first enzyme, said first substrate being labeled with a first fluorphore,
providing a second substrate for a second enzyme, said second substrate being labeled with a second fluorphore,
exposing the labeled substrates to the sample to allow the first and second enzymes present in the sample to interact with respective first and second fluorophore-labeled substrates to form respective first and second fluorphore-labeled substrate fragments; detecting the presence of said fluorphore-labeled substrate fragments
2. A method according to paragraph 1, wherein the first and/or second substrate is a protein or polypeptide.
3. A method according to paragraph 2, wherein the protein or polypeptide is selected from the group consisting of: gelatin, porcine thyroglobulin, collagen, immunoglobulin or bovine serum albumin.
4. A method according to any preceding paragraph, wherein at least one of the substrates is carried on a support.
5. A method according to paragraph 4, wherein the support is a solid phase support comprising; glass or sol gel beads or cellulose fibres.
6. A method according to paragraph 4 or 5, wherein the beads are magnetisable.
7. A method according to preceding paragraph, wherein the first and second enzyme is selected from the enzyme group consisting of: protease, cellulase, lipase, □-amylase or collagenase
8. A method according to paragraph 7, wherein the protease is selected from the group consisting of: subtilisin-type, trypsin, papain, esperase or alcalase.
9. A method according to any preceding paragraph, wherein the first and second fluorophore is fluorescein, rhodamine, Texas Red® or lucifer yellow.
10. A method according to any preceding paragraph, wherein the sample is air.
11. A method according to any preceding paragraph, wherein the enzyme(s) derived from the sample simultaneously contact the first substrate and the second substrate.
12. A method according to any preceding paragraph, wherein the enzyme(s) derived from the sample sequentially contact the first substrate and the second substrate.
13. A method according to paragraph 11 or 12, wherein the second substrate is a substrate for a protease.
14. A vessel for use in the simultaneous detection of the presence of at least two enzymes in a sample, said vessel comprising a first substrate for a first enzyme, said first substrate being labeled with a first fluorphore, and a second substrate for a second enzyme, said second substrate being labeled with a second fluorphore.
15. A vessel according to paragraph 14, wherein at least one of the substrates is carried on a support.
16. A vessel according to paragraph 15, wherein the support is a solid phase support comprising; glass or sol gel beads or cellulose fibres
17. A vessel according to paragraph 16, wherein the vessel contains a heterogeneous mixture of the first and second substrates.
18. A vessel according to paragraph 16, wherein the vessel contains a layer of the first substrate and a layer of the second substrate.
19. Apparatus for use in the simultaneous detection of at least two enzymes in a sample, said apparatus comprising a vessel comprising a first substrate for a first enzyme, said substrate being labeled with a first fluorophore and a second substrate for a second enzyme, said second substrate being labeled with a second fluorophore, said apparatus further comprising detecting means for detecting fluorescent substrate fragments.
20. Apparatus according to paragraph 19, wherein the first and second substrates are layered within the vessel and the layer of the second substrate, being a substrate for a protease, is located adjacent to said detecting means.
21. Apparatus for use in the simultaneous detection of at least two enzymes in a sample, said apparatus comprising a first vessel comprising a first substrate for a first enzyme, said substrate being labeled with a first fluorophore, said first apparatus being connectable to a second vessel, wherein the second vessel comprises a second substrate for a second enzyme, said substrate being labeled with a second fluorophore, said apparatus further comprising detecting means for detecting fluorescent substrate fragments.
22. Apparatus according to paragraph 21, wherein the substrate in the second vessel is a protease, and the second vessel is located adjacent to the detecting means.
23. A method, vessel or apparatus as substantially herein described with reference to the accompanying example and FIG. 1.

1. A method for simultaneously detecting the presence or absence of at least two enzymes in a sample, said method comprising the steps of;
(i) exposing (a) a first substrate for a first enzyme, said first substrate being labeled with a first fluorphore and (b) a second substrate for a second enzyme, said second substrate being labeled with a second fluorphore to the sample to allow the first and second enzymes present in the sample, if present, to interact with respective first and second fluorophore-labeled substrates to form respective first and second fluorphore-labeled substrate fragments;
(ii) detecting the presence of said fluorphore-labeled substrate fragments

2. The method of claim 1, wherein the method is for the detection of levels of at least two air-borne enzymes and which comprises the following steps:
(i) prior to exposing the substrates to the sample, obtaining a first sample of atmospheric air; and
(ii) capturing any enzyme particles which are present in the first sample.

3. The method of claim 2, further comprising mixing the captured enzyme particles with a liquid to form the sample.

4. The method of claim 3, wherein the liquid is an aqueous buffer.

5. The method of claim 4, further comprising conveying the sample from an area in which first sample is mixed with the liquid to a reaction area in which the sample is exposed to the substrates.

6. The method of claim 1, wherein the step of exposing the sample to the substrates is carried out for a period of time sufficient to enable interaction between the first and/or the second enzyme with the respective substrate to form fluorophore label substrate fragment.

7. The method of claim 1, wherein the step of detecting involves detecting a signal emitted by the first and/or second fluorophore labeled substrate fragment.

8. The method of claim 1, wherein the first and/or second substrate is a protein or polypeptide.

9. The method of claim 8, wherein the protein or polypeptide is selected from the group consisting of gelatin, porcine thyroglobulin, collagen, an immunoglobulin or fragment thereof and bovine serum albumin.

10. The method of claim 1, wherein at least one of the substrates is carried on a support.

11. The method of claim 10, wherein the support is a solid phase support.

12. The method of claim 10, wherein the support is produced from or comprises glass, sol gel beads, cellulose fibres or silica particles.

13. The method of claim 10, wherein the support is magnetisable.

14. The method of claim 1, wherein the first and/or the second enzyme is selected from the enzyme group consisting of protease, cellulase, lipase, σ-amylase or collagenase.

15. The method of claim 14, wherein the protease is selected from the group consisting of subtilisin-type, trypsin, papain, esperase and alcalase.

16. The method of claim 1, wherein the first and/or the second fluorophore is selected from fluorescein and derivatives thereof, rhodamine, Texas Red® and lucifer yellow.

17. The method of claim 1, wherein the sample is derived from a first sample of air.

18. The method of claim 14, wherein one or both of the first and the second substrate is a substrate for a protease.

19. The method of claim 1, which comprises first exposing the sample to one of the first or second substrates and subsequently exposing the sample to the other of the first or second substrates.

20. The method of claim 18, wherein at least one of the enzymes being monitored is a protease and at least one of the first substrate and second substrate is a substrate for protease.

21. The method of claim 20, wherein the enzyme is a subtilisin-type enzyme and the substrate is α-amylase.

22. The method of claim 1, which comprises exposing the sample to the first substrate and the second substrate at the same time.

23. A vessel for use in the simultaneous detection of the presence of at least two enzymes in a sample, said vessel comprising, in use, a first substrate for a first enzyme, said first substrate being labeled with a first fluorphore, and a second substrate for a second enzyme, said second substrate being labeled with a second fluorphore.

24. The vessel of claim 23, which comprises a support on which at least one of the first and second substrates is supported.

25. The vessel of claim 23, wherein the support is a solid phase support comprising glass or sol gel beads silica particles or cellulose fibres

26. The vessel of claim 25, wherein the vessel contains a heterogeneous mixture of the first substrate and the second substrate.

27. The vessel of claim 26, wherein the vessel contains at least one layer which is substantially composed of the first substrate and at least one layer which is substantially composed of the second substrate.

28. An apparatus for use in the simultaneous detection of at least two enzymes in a sample said apparatus comprising a vessel comprising a first substrate for a first enzyme, said substrate being labeled with a first fluorophore and a second substrate for a second enzyme, said second substrate being labeled with a second fluorophore, said apparatus further comprising detecting means for detecting fluorescent substrate fragments.

29. The apparatus of claim 28, wherein the first and second substrates are layered within the vessel.

30. The apparatus of claim 29, wherein when one of first substrate and second substrate is a substrate for a protease enzyme, it is positioned such that is downstream from the other substrates.

31. An apparatus for use in the simultaneous detection of at least two enzymes in a sample, said apparatus comprising a first vessel comprising a first substrate for a first enzyme, said substrate being labeled with a first fluorophore, said first apparatus being connectable to a second vessel, wherein the second vessel comprises a second substrate for a second enzyme, said substrate being labeled with a second fluorophore, said apparatus further comprising detecting means for detecting fluorescent substrate fragments.

32. The apparatus of claim 31, wherein when one of the first substrate or second substrate is a substrate for a protease enzyme, it is situated in the vessel downstream from the other vessel and is located adjacent to the detecting means.

33. The method of claim 19, wherein at least one of the enzymes being monitored is a protease and at least one of the first substrate and second substrate is a substrate for protease.

34. The vessel of claim 24, wherein the support is a solid phase support comprising glass or sol gel beads, silica particles or cellulose fibres
